(19) United States
(12) Patent Application Publication
(10) Pub. No.: US 2001/0022181 A1
(43) Pub. Date: Sep. 20, 2001

(54) Anesthesia ventilator with automatic circuit switch

(76) Inventors: Jean-Philippe Masson, Chaville (FR); Sebastien Kitten, Saulx Les Chartreux (FR)

Correspondence Address: YOUNG & THOMPSON, 745 SOUTH 23RD STREET 2ND FLOOR, ARLINGTON, VA, 22202

(21) Appl. No.: 09/761,767

(22) Filed: Jan. 18, 2001

(30) Foreign Application Priority Data
Jan. 19, 2000 (FR) 0000636

(51) Int. Cl. G08B 3/00; G08B 5/00; A62B 9/00; A61M 16/00; A62B 27/00; A61M 15/00; A61M 16/10; A62B 7/10; A62B 19/00; A62B 23/02

(52) U.S. Cl. 128/203.12; 128/205.12; 128/202.22; 128/205.23; 128/205.28

(57) ABSTRACT

The invention relates to a device for dispensing gases to a patient, in particular anesthetic gases, with reinhalation of the gas exhaled.

It comprises:

a first gas circulation conduit (10), a switch device (12) comprising an inlet (14) connected to said first conduit (10), and first and second outlets (16, 18), a second gas circulation conduit portion (30), a removable gas filtration unit (20), said unit comprising a gas inlet (24) which can be connected to the first outlet of the switch device, and a gas outlet (28) which can be connected to said second conduit, a branch conduit (34) whose first end is connected to the second outlet (18) of the switch device and whose other end is connected to said second conduit, detection means (36) for detecting the presence of said filter unit in the circuit; and control means (38) controlling the switch device (12) so as to cause said switch device to assume a first state in which its inlet (14) is connected to its first outlet (16), and, in the absence of detection information, to cause said switch device to assume a second state in which its inlet (14) is connected to its second outlet (18).

The present invention relates to a device for dispensing gases to a patient, in particular anesthetic gases, with reinhalation of the gas exhaled by the patient.

During a surgical intervention performed under anesthesia, it is necessary to supply the patient with a gas mixture which in most cases comprises oxygen, an anesthetic gas, and in most cases also air.

In a number of cases, for example in the case described in European Patent Application 835 672, the device for dispensing gas to the patient has a closed circuit, that is to say that the gas exhaled by the patient is reinhaled by said patient. For the user, an installation of this type has the advantage of limiting the quantity of gas since the latter is reduced after initial filling to compensate for the losses due to leaks and consumption of gases by the patient. It will be appreciated that in such an installation it is of course necessary to filter the recycled gas in order to remove from it carbon dioxide in particular.

The medium used for filtration of the carbon dioxide must in most cases be changed, possibly during anesthesia if the surgical intervention is relatively protracted. This generally necessitates removal of the container, filled with the filter medium, which is mounted in series in the line of flow of the gases administered to or issuing from the patient. Removal of this container can be done in different ways.

Depending on whether the gas filtration system is situated on the inhalation branch or on the exhalation branch of the circuit with reinhalation, several solutions have already been implemented.

In the case where the purification and filtration system is on the inhalation branch, it is possible either to completely shut off gas insufflation by opening the circuit with a view to changing the filter material, or to use a mechanical member which, by manual activation, makes it possible, before removal of the container filled with the filter material, to create a closed circuit with reinhalation of the gas, but this circuit is deprived of filtering.

The solutions set out above all entail risks or inconveniences for the user. In the first solution, the patient is not ventilated during change of the filter medium, and if the enclosure containing this medium breaks, the machine cannot be used. Finally, opening the circuit causes a modification of the gas concentrations previously established in this circuit, and also the escape, to atmosphere, of the anesthetic gases, which can pollute the operating theater.

In the second solution, the user has to perform a supplementary manipulation before and after removing the container filled with the filter material in order, in the first instance, to continue the closed-circuit ventilation with reinhalation but without purification of the gas before returning to a closed circuit with reinhalation and with purification of the gas by filtration.

In the case where the purification system is placed on the exhalation branch, the two possible solutions are the following:

Either the use of a manual system ensuring maintenance of the closed circuit, without the latter including a filter system when the latter is disassembled. This entails the same difficulties as in the case of the inhalation branch;

Or the removal of the filter system causes opening of the exhalation branch with maintenance of ventilation of the patient. The patient is at all times connected to the machine for insufflation, but the exhaled gases are eliminated completely without any recycling. Use will considerably increase the quantity of gas delivered by the machine.

In this latter case, opening the circuit has the same consequences as in the first solution in terms of the concentrations of gas established in the circuit and the possible pollution of the operating theater. Moreover, it requires the operator to increase the gas flowrates in order to function in a circuit without reinhalation.

The subject of the present invention is a device for dispensing gases to a patient, in particular anesthetic gases, with reinhalation of the exhaled gas, which device makes it possible to avoid the abovementioned disadvantages by permanently ensuring ventilation of the patient without necessitating any particular operation on the part of the operator during removal of the container filled with the filter material when this is to be changed.

To achieve this aim according to the invention, the device for dispensing gases to a patient, in particular anesthetic gases, with reinhalation of the gas exhaled, comprises:

a first gas circulation conduit portion,

a switch device comprising an inlet connected to said first conduit portion, and first and second outlets,

a second gas circulation conduit portion which has two inlets,

a removable gas filtration unit, said unit comprising a gas inlet which can be connected to the first outlet of the switch device, and a gas outlet which can be connected to an inlet of said second conduit portion,

a branch conduit whose first end is connected to the second outlet of the switch device and whose other end is connected to the second inlet of said second conduit portion,

detection means for detecting the presence of said filter unit in the circuit and for supplying detection information in the event of said unit being present; and

control means controlling the switch device so as to cause said switch device, in response to the detection information supplied by the detection means, to assume a first state in which its inlet is connected to its first outlet, and, in the absence of detection information, to cause said switch device to assume a second state in which its inlet is connected to its second outlet.

It will be appreciated that by means of the device according to the invention, when it is necessary to remove the filter unit from the ventilation circuit, this situation is detected automatically and the switch device is also controlled automatically to ensure reinhalation, by the patient, of the exhaled gas by way of the branch conduit.

As regards the detection means and control means of the switch device, these can be electrical or mechanical, or of any appropriate type.

According to a preferred embodiment corresponding to mechanical detection and mechanical control, the device is characterized in that the switch device comprises an enclosure provided with said gas inlet and said first and second gas outlets, a movable shutter element inside said enclosure, able to close off said first outlet or said second outlet, and return means for returning the shutter element to a first position corresponding to closure of said first outlet, and in that the detection and control means comprise a mechanical member connected to the inlet of said filter unit, and when said filter unit is present, said mechanical member is able to bring said shutter element to a second position in which it closes off said second outlet.

It will be appreciated that in this embodiment the detection and the control of the switch are very simple and very reliable since it is a mechanical member directly which, after removal of the filter unit, causes the change of state of the switch so that its outlet is connected to the branch conduit.

Other characteristics and advantages of the invention will become clear on reading the following description of several embodiments of the invention which are given by way of nonlimiting example. In the description, reference is made to the attached figures, in which:

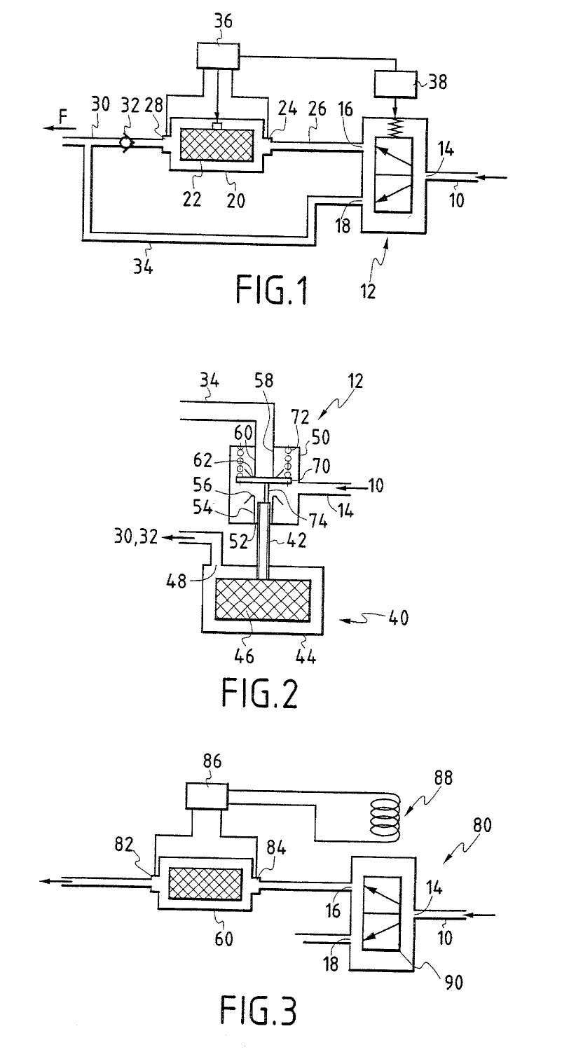

FIG. 1 is a block diagram showing the whole of the device;

FIG. 2 is a partial view showing an embodiment in which the presence of the filter unit is detected by mechanical means; and

FIG. 3 is a partial view showing a system of electrical detection.

A simplified description of the overall device for dispensing gas to a patient will now be given, referring first to FIG. 1.

This figure shows a first conduit portion 10 carrying the gas, for example the anesthetic gas. The conduit 10 is connected to a flow switch 12 and more precisely to its single inlet 14. The function of the switch 12 is to connect the inlet 14 either to a first outlet 16 or to a second outlet 18. The first outlet 16 is connected to a container 20 which contains a material 22 for filtering the gas exhaled by the patient in order to absorb the carbon dioxide from it. The filter container 20 includes a gas inlet 24 connected to the outlet 16 via a conduit element 26. The connection between the conduit 26 and the inlet 24 of the container 20 is removable so that, as will be explained below, it is possible to disconnect the filter container from the installation with a view to changing the filter material 22. The gas outlet 28 of the container 20 is connected to a first inlet 30a of a second gas circulation conduit portion 30 via a nonreturn valve 32 which allows the gas to circulate only in the direction indicated by the arrow F. The installation also includes a branch conduit 34 which connects the second outlet 18 of the switch 12 directly to the second inlet 30b of the second conduit portion 30 downstream of the nonreturn valve 32.

The installation also includes a detection circuit 36 with which it is possible to detect the actual presence of the container 20 in the circuit. The detection means 36 are connected to means 38 controlling the switch 12.

In normal functioning, the filter container 20 is present in the circuit. This presence, confirmed by the means 36, is transmitted to the control means 38 of the switch which occupies a first state in which its inlet 14 is connected to its outlet 16. Thus, the flow of gas passes through the filter container and, purified, leaves the latter and enters the second conduit portion 30.

When the user, for example the anesthetist, wishes to change the filter container, he disconnects the container from the conduits 26 and 30. This situation is automatically detected by the detection means 36 and is transmitted to the control means 38 of the switch 12. The latter assumes its second state which brings the inlet 14 into communication with the outlet 18. Thus, the new ventilation circuit for the patient is formed by the conduit portion 10, the branch conduit 34 and the conduit portion 30.

When the user wishes to fit a new filter container 20, the latter is reconnected to the conduits 26 and 30. The presence of a new filter container is detected at 36 and the control means 38 move the switch 12 to its first state, which returns the ventilation circuit to its state of normal functioning, that is to say with filtering of the exhaled air.

As has already been explained, the means 36 for detecting the presence of the filter unit and the means 38 controlling the state of the switch 12 can preferably be electrical or mechanical. A mechanical embodiment of these two units will be described with reference to FIG. 2.

In said FIG. 2, the filter container is labeled 40 and the mechanical switch is labeled 12. The container 40, formed by an enclosure 44 containing the filter material 46, includes an outlet 48 connected to the second gas conduit portion 30. The gas inlet of the container 40 is formed by a flue 42 which projects from this container 40.

The mechanical switch 12 comprises an enclosure 50 provided with a first opening 52 continued internally by a sleeve 54 which ends in a valve seat 56. The sleeve 54 is configured in such a way that it can receive the end of the flue 42 in a leaktight manner. The enclosure 50 of the switch includes a second opening 58 continued via an internal sleeve 60 forming a second valve seat 62 opposite the first valve seat 56. The sleeve 58 is connected to the branch conduit 34. Finally, the enclosure 50 of the switch includes the gas inlet 14 connected to the gas conduit 10. The switch 12 also includes a movable shutter element 70 which can assume two positions, bearing either on the seat 56 or on the seat 62. A return spring 72 tends to maintain the shutter element 70 on the seat 56. Guide means (not shown) are associated with the movable shutter element 70.

In this embodiment, the end of the flue 42 of the filter container 40 is continued via a rod 74 which, when the container 40 is engaged in the sleeve 54, acts on the shutter element 70 in order to maintain the latter pressed flat against the seat 62, compressing the return spring 72. In this position, the gas enters the switch 12 via the inlet 14 and passes into the flue 42 of the filter container since the sleeve 60 is closed off by the shutter element 70. By contrast, when the filter container is removed, the return spring 72 applies the shutter element 70 to the seat 56 and thereby frees the opening of the sleeve 60. The gas passing into the switch 12 via the inlet 14 leaves it via the sleeve 60 and the branch conduit 34.

This mechanical solution has the advantage of being extremely reliable since it is mechanical members directly which ensure detection of the presence of the filter container and transmission of the detection information, and cause the switch to assume its corresponding state.

It will be appreciated that in this embodiment the detection information consists in detection of the presence or absence of the rod 74 acting on the shutter element 70.

FIG. 3 illustrates an electrical system for detection and control of the switch.

Electrical sensors 82 and 84 are mounted in the two zones of connection of the filter container 60 with the conduits. When one of the connections is disconnected, the corresponding sensor transmits a signal to the processing circuit 86.

The processing circuit 86 applies to a control coil 88 a current which is able to move the slide 90 of the switch 80 into a position in which it brings the inlet 14 into communication with the outlet 18. When the filter enclosure 60 is again coupled to the two conduits, the sensors 82 and 84 send a signal to the processing circuit 86. The circuit 86 then applies to the control coil 88 an electric current which is able to move the slide 90 into a position in which it brings the inlet 14 of the switch into communication with its outlet 16.

The filter unit 20, the switch 12 and the branch conduit 34 can just as well be fitted in the inhalation branch as in the exhalation branch, the conduit portions 10 and 30 then constituting either the inhalation conduit for conveying the gas mixture to the patient, or the exhalation conduit for collecting the gas mixture exhaled by the patient and for recycling it.

The device for dispensing gas can constitute either a closed circuit for anesthesia or a semi-closed circuit for anesthesia.

1. A device for dispensing gases to a patient, in particular anesthetic gases, with reinhalation of the gas exhaled, comprising:
a first gas circulation conduit portion (10, 12),
a switch device comprising an inlet (14) connected to said first conduit portion (10), and first and second outlets (16, 18),
a second gas circulation conduit portion (30) which has two inlets (30a, 30b),
a removable gas filtration unit (20), said unit comprising a gas inlet (24) which can be connected to the first outlet of the switch device, and a gas outlet (28) which can be connected to an inlet of said second conduit portion,
a branch conduit (34) whose first end is connected to the second outlet (18) of the switch device and whose other end is connected to the second inlet (30b) of said second conduit portion,
detection means (36) for detecting the presence of said filter unit in the circuit and for supplying detection information in the event of said unit being present; and
control means (38) controlling the switch device (12) so as to cause said switch device, in response to the detection information supplied by the detection means, to assume a first state in which its inlet (14) is connected to its first outlet (16), and, in the absence of detection information, to cause said switch device to assume a second state in which its inlet (14) is connected to its second outlet (18).

2. The device as claimed in claim 1, wherein said second conduit portion (30) is provided with nonreturn means (32), and wherein the second end of the branch conduit (34) is connected to the inlet of said second conduit portion (30) downstream of said nonreturn means.

3. The device as claimed in either of claims 1 and 2, wherein said detection means (82, 84, 86) and said control means (88) of the switch device (12) are electrical.

4. The device as claimed in either of claims 1 and 2, wherein said detection means and said control means of the switch device are mechanical.

5. The device as claimed in claim 4, wherein said switch device (12) comprises an enclosure (50) provided with said gas inlet (14) and said first (52) and second (58) gas outlets, a movable shutter element (70) inside said enclosure, able to close off said first outlet or said second outlet, and return means (72) for returning the shutter element to a first position corresponding to closure of said first outlet, and wherein the detection and control means comprise a mechanical member (74) connected to the inlet (42) of said filter unit (40), and, when said filter unit is present, said mechanical member is able to bring said shutter element (70) to a second position in which it closes off said second outlet.

6. The device as claimed in any of claims 1 through 5, wherein said first (10) and second (30) conduit portions constitute the inhalation branch of said device for dispensing gases.

7. The device as claimed in any of claims 1 through 5, wherein said first (10) and second (30) conduit portions constitute the exhalation branch of said device for dispensing gases.

8. The device as claimed in any of claims 1 through 7, wherein said dispensed gas is an anesthetic gas or anesthetic gas mixture, and wherein said dispensing device is of the closed circuit type.

9. The device as claimed in any of claims 1 through 7, wherein said dispensed gas is an anesthetic gas or anesthetic mixture, and wherein said dispensing device is of the semi-closed circuit type.
